(19) United States
(12) Patent Application Publication
(10) Pub. No.: US 2026/0295101 A1
(43) Pub. Date: Oct. 1, 2026

(54) STERILISATION METHOD FOR A HOLLOW FIBRE MEMBRANE FILTER

(71) Applicant: FRESENIUS MEDICAL CARE DEUTSCHLAND GMBH, Bad Homburg (DE)

(72) Inventors: Michael PAUL, Bad Homburg (DE); Jeremias GLESER, Bad Homburg (DE); Eric-Peter SCHU, Bad Homburg (DE)

(73) Assignee: FRESENIUS MEDICAL CARE DEUTSCHLAND GMBH, Bad Homburg (DE)

(21) Appl. No.: 19/477,795

(22) PCT Filed: Apr. 26, 2024
(86) PCT No.: PCT/EP2024/061565

§ 371 Date: Oct. 23, 2025

(30) Foreign Application Priority Data
Apr. 28, 2023 (DE) 10 2023 204 005.0

(51) Int. Cl. A61L 2/07; A61L 101/48; A61L 103/09; A61L 103/15

(52) U.S. Cl. CPC A61L 2/07; A61L 2101/48; A61L 2103/09; A61L 2103/15

(57) ABSTRACT

The present invention relates to a method for sterilizing a hollow fiber membrane filter with a sterilizing fluid, such as Water or water vapor, comprising a rinsing step, a sterilization step, and a leakage test to ensure enhanced safety of sterility of the hollow fiber membrane filter.

The subject of the present application is a method of sterilizing hollow fiber membrane filters used for extracorporeal blood treatment of patients with kidney disease.

Hollow fiber membranes used for blood purification are often made of polysulfone (PSU) and polyvinylpyrrolidone (PVP), as these materials have been found to be preferentially hemocompatible and are therefore preferred from a medical point of view in blood treatment, especially in hemodialysis.

In the manufacture of such hollow fiber membranes, a spinning mass is extruded through a nozzle to form a hollow yarn and is introduced into a precipitation bath, usually consisting of water. To form the pore structure, an aqueous precipitant is used that contains an aprotic solvent such as dimethylacetamide, N-metyhlpyrrolidone, dimethylformamide or dimethyl sulfoxide. The obtained hollow fiber membrane is then passed through rinsing baths, dried and taken up on a reel. The Hollow fiber membranes can be taken out from the reel in the form of hollow fiber membrane bundles. For the construction of hollow fiber membrane filters, such hollow fiber membrane bundles are then placed in a housing, preferably a cylindrical housing. The ends of the hollow fiber membrane bundle are embedded in a potting compound and the open ends of the hollow fibers are exposed. The potting compound forms a sealing area between the interior of the hollow fiber membranes, the housing, and the space surrounding the hollow fiber membranes. A first compartment is thus formed in the hollow fiber membrane filter, which includes the inflow and outflow areas of the ends of the hollow fiber membrane bundle and also the interior of the hollow fiber membranes. Accordingly, a second compartment is formed in the space between the hollow fiber membranes and between the housing wall and the hollow fiber membranes. Fluid inlets on the housing of the hollow fiber membrane filter allow liquids and fluids to be supplied to and discharged from the first and/or second compartment of the hollow fiber membrane filter.

Hollow fiber membrane filters provided for extracorporeal blood treatment typically include first and second fluid inlets on the first compartment of the filter module, and first and second fluid inlet ports on the second compartment of the filter module. Fluids, in particular liquids or gases, can thus be supplied or discharged via the first inlet of a compartment of the hollow fiber membrane filter depending on the direction of flow or via the second inlet of a compartment of the hollow fiber membrane filter corresponding to the direction of flow.

For hollow fiber membrane filters intended for medical use, especially for blood treatment of patients with kidney disease, the hollow fiber membrane manufacturing process and filter construction are usually followed by one or more rinsing and sterilization steps to clean and sterilize the hollow fiber membranes for the medical use.

According to the prior art, processes are known in which the hollow fiber membranes in hollow fiber membrane filters are subjected to rinsing and sterilization steps. In particular, heat sterilization with water or water vapor is a well-known sterilization process for hollow fiber membranes and hollow fiber membrane filters. Heat sterilization is understood to mean sterilization with fluids (for example water or water vapor or mixtures thereof) above a temperature of 100° C. Heat sterilization with predominantly pure water vapor is also called steam sterilization.

A corresponding process for sterilizing dialyzers is described in DE 39 36 785C1. According to this procedure, the dialyzers are first subjected to a rinsing process and then to a sterilization process. During the sterilization process, the dialyzer is rinsed with water or water vapor heated to over 121° C. The heat sterilization carried out in according to DE 39 36 785 C1 has proven to be superior to other sterilization methods in terms of technical equipment and process technology.

WO 2018/204498 A9 describes a sterilization process for hollow fiber membrane filters comprising hollow fiber membranes of polysulfone and polyvinylpyrrolidone. In the sterilization process described, a process step is used in which water vapor is passed across the membrane wall by adjusting the pressure ratios in the first and second compartments of the hollow fiber membrane filter.

The processes known in the prior art are designed in such a way that further process steps follow the actual sterilization step. In particular, in these procedures, the leakage test of the hollow fiber membrane filter was performed downstream of the sterilization step. The leakage test carried out during steam sterilization of hollow fiber membrane filters is usually performed as a so-called “bubble point test”. In this test, the second compartment of the hollow fiber membrane filter defined above is filled with sterile compressed air or, alternatively, sterile compressed air flows through it. The second compartment of the hollow fiber membrane filter is filled or flown through with highly purified water. Through an upstream step, the pores of the hollow fiber membranes are filled with water. The pressure of the sterile compressed air in the “bubble point test” is selected such that the surface tension of the water in the pores is not overcome and the water remains in the pores. However, if leakage exists between the first compartment and the second compartment of the hollow fiber membrane filter, at the set pressure the sterile compressed air will leak into the first compartment and be expelled from the first compartment with the highly purified water flowing through it. An air bubble detector connected to an outlet of the first compartment of the hollow fiber membrane filter can detect the leaked sterile compressed air, and the hollow fiber membrane filter can be sorted out accordingly in the process.

The disadvantage described in the prior art is that the leakage test is carried out after the actual sterilization step. There is therefore a need to further improve the quality of the sterility of the filter. In addition, however, the integrity of a hollow fiber membrane filter and thus also the performance of the leakage test is of utmost importance, since hollow fiber membrane filters that show leakage due to its production are not allowed to be used in medical applications.

### OBJECTIVE OF THE INVENTION

With regard to the problems prevailing in the prior art, it was an object to provide a process for sterilizing hollow fiber membrane filters that is based on the principle of steam and heat sterilization, and which has a high sterilization quality, but also ensures the integrity of a hollow fiber membrane filter.

### SUMMARY OF THE INVENTION

The object of the present invention is solved by a method according to claim 1. Claims 2 to 14 represent preferred embodiments of the method.

### DESCRIPTION OF THE INVENTION

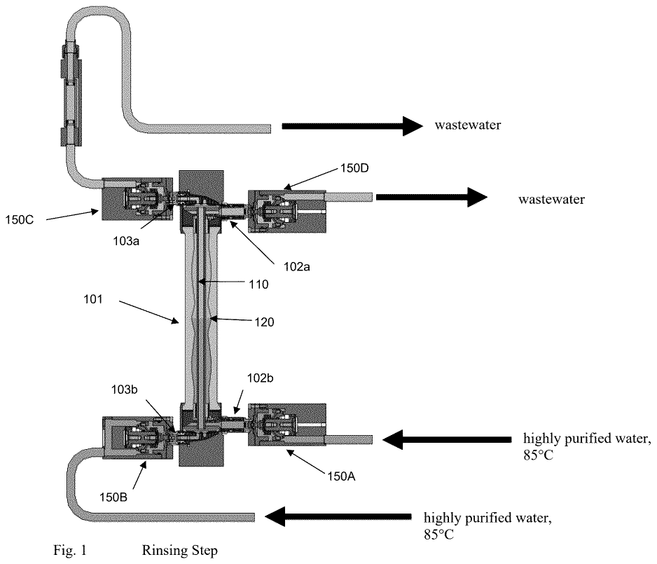

In the following, the present invention is described with reference to individual embodiments and is illustrated with reference to FIG. 1 to 4.

The present invention relates to a method for sterilizing a hollow fiber membrane filter having a plurality of hollow fiber membranes sealed at the ends in the housing of the hollow fiber membrane filter so that a first compartment involving the interior of the hollow fiber membranes and a second compartment involving a space between the hollow fiber membranes is formed, wherein the hollow fiber membrane filter has at least two fluid inlets connected to the first compartment and at least two fluid inlets connected to the second compartment, and wherein the fluid inlets are configured to be connected to a sterilization device. The method comprises at least the steps of:
rinsing the hollow fiber membrane filter with a fluid, in particular water, wherein the rinsing fluid is passed through the first and second compartments of the hollow fiber membrane filter via a selection of the fluid inlets, carrying out a leakage test, sterilizing the hollow fiber membrane filter with a sterilizing fluid, in particular heated water or water vapor, wherein the sterilizing fluid is passed through the first and second compartments of the hollow fiber membrane filter via a selection of the fluid inlets, characterized in that the leakage test is performed prior to the sterilizing step.

In a further embodiment, the method according to the invention is characterized in that, in the leakage test, sterile compressed air is introduced into, in particular is passed through, the second compartment of the hollow fiber membrane filter, and highly purified water is introduced into, in particular is passed through, the first compartment of the hollow fiber membrane filter, and the pressure in the second compartment is higher than in the first compartment.

According to the method of the invention, the sterilization step is performed after the leakage test. This ensures that after the sterilization step no further rinsing steps, e.g. in the course of a leakage test, occur, which could cause the hollow fiber membrane filter to become contaminated with germs again.

FIG. 1 shows a schematic illustration of the rinsing step of the method according to the invention. Shown is a hollow fiber membrane filter 101 having two fluid inlets 102a, 102b that provide access to the second compartment 120 of the hollow fiber membrane filter 101. Schematically, in FIG. 1 only one of a multitude of hollow fiber membranes is shown. At 103a, 103b, fluid inlets are shown forming an access to the first compartment 110 of the hollow fiber membrane filter. Fluid inlets 102a, 102b, 103a, 103b are connected to a sterilization device via connection devices 150A, 150B, 150C, 150D not shown in FIG. 1. A flow of highly purified water is provided via the sterilization device and is introduced into the second and first compartments 120, 110 of the hollow fiber membrane filter 101 via the connection devices 150A, 150B and the fluid inlets 102b, 103b. The first and second compartments 110, 120 are flowed through by highly purified water according to the embodiment of FIG. 1. The hollow fiber membrane filter is thus preconditioned for the subsequent sterilization step. Within the meaning of the present application highly purified water as described in the European Pharmacopoeia PH. EUR. 04/2017:0169″ is used.

The temperature of the in flowing highly purified water is 85° C. according to the embodiment shown. In general, however, depending on the design of the hollow fiber membrane filter, other temperatures between 50 to 120° C., 50 to 95° C. or 60 to 90° C. or 65 to 90° C. or 70 to 90° C. of the highly purified water can be set in the rinsing step. The highly purified water exits the first and second compartments 110, 120 via fluid inlets 103a, 102a and connection devices 150C, 150D and is discharged as wastewater.

The duration of the rinsing step is preferably 40 seconds(s). Depending on the design of the hollow fiber membrane filter, alternative durations of the rinsing step can be used, e.g. 20 to 60 s, 30 to 60 s, 35 to 50 s, or 35 to 45 s.

During the rinsing step, all air is expelled from the hollow fiber membrane filter, that means that the air in the first and second compartments 110, 120 of the hollow fiber membrane filter and in particular also the air from the pores of the membrane wall of the hollow fiber membranes are expelled.

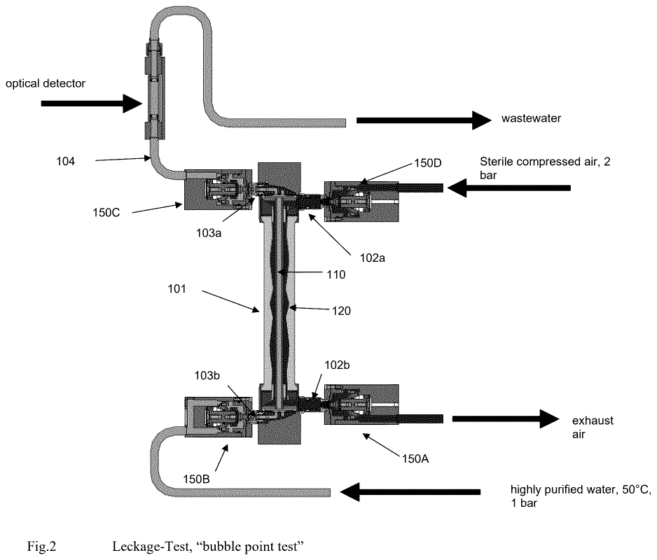

FIG. 2 shows a schematic illustration of the leakage test step of the method according to the invention. In the embodiment shown, the leakage test is carried out as a so-called “bubble point test”. The arrangement of the hollow fiber membrane filter, the connection devices 150A, 150B, 150C, 150D in conjunction with the sterilization device (not shown in FIG. 2) is the same as in FIG. 1. In the schematically illustrated process step of the leakage test, highly purified water is provided via the sterilization device and is introduced into the first compartment 110 of the hollow fiber membrane filter via the connection device 150B and the fluid inlet 103b. In this embodiment, the highly purified water has a temperature of 50° C. and a pressure of 1 bar. The temperature can vary depending on the design of the hollow fiber membrane filter and can range from 30 to 70° C., 40 to 60° C. or 45 to 55° C. The pressure can be correspondingly between 0.5 to 1.5 bar or between 0.9 to 1.1 bar. Sterile compressed air flows into the second compartment of the hollow fiber membrane filter via the connection device 150D and the fluid inlet 102a. The pressure of the sterile compressed air in the embodiment shown is 2 bar and can vary between 1.5 and 3.5 bar depending on the design of the hollow fiber membrane filter and the pore size in the hollow fiber membranes. The highly purified water is passed through the first compartment 110 and is discharged via the fluid inlet 103a and the connection device 150C. The sterile compressed air is passed through the second compartment 120 of the hollow fiber membrane filter 101 and is discharged via the fluid inlet 102b and the connection device 150 A. In the embodiment shown, sterile compressed air and highly purified water are passed through the respective second and first compartments 120, 110 of the hollow fiber membrane filter using the counterflow principle. Provided there is leakage between the first compartment and the second compartment, sterile compressed air will leak from the second compartment 120 into the first compartment 110 and can be detected. Accordingly, an optical detector is attached to the wastewater conduit 104, wherein the wastewater conduit is attached to the connection device 150C and is in fluid communication with the first compartment 110 of the hollow fiber membrane filter 101. The compressed air that passes into the first compartment in the event of leakage is expelled as air bubbles with the highly purified water from the first compartment of the hollow fiber membrane filter and can be detected optically in the wastewater line by appropriate detectors.

Provided that it has been determined that the hollow fiber membrane filter is free of leakage, the second compartment 120 of the hollow fiber membrane filter 101 is filled with air after the leakage test, and the first compartment is filled with highly purified water. In addition, the pores of the membrane wall of the hollow fiber membranes are also filled with highly purified water.

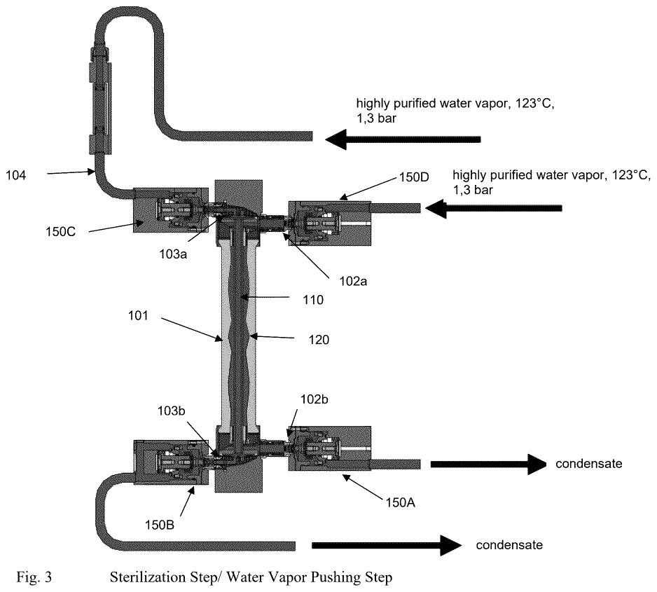

FIG. 3 shows a schematic illustration of the sterilization step of the method according to the invention. The arrangement of the hollow fiber membrane filter, the connection devices 150A, 150B, 150C, 150D in conjunction with the sterilization device (not shown in FIG. 3) is the same as in FIG. 1 and FIG. 2. In the sterilization step, highly purified water vapor is introduced into the first compartment 110 and the second compartment 120, respectively, of the hollow fiber membrane filter 101 via the connection devices 150C and 150D and the fluid inlets 103a and 102a. The highly purified water vapor is provided via the sterilization device and, according to the embodiment shown, has a temperature of 123° C. and a pressure of 1.3 bar. However, depending on the design of the hollow fiber membrane filter, other pressures and temperatures may be used to ensure optimum sterilization results. In particular, pressures of 1.1 to 3 bar or 1.1 to 2.5 bar or 1.1 to 2 bar, or 1.1 to 1.8 bar or 1.2 to 1.6 bar can also be used for the sterilization step. Alternative temperatures of the highly purified water vapor range from 105 to 150° C. or from 110 to 140° C. or from 115 to 130° C. The highly purified water vapor is passed through the first compartment and the second compartment 110, 120 of the hollow fiber membrane filter, and the first compartment and the second compartment are thereby sterilized. As the highly purified water vapor passes through the first and second compartments, the highly purified water vapor cools, causing some of the highly purified water vapor to condense. In the technical process, the mixture of highly purified water vapor and condensed water is referred to as condensate and is discharged from the second compartment and the first compartment via fluid inlets 102b, 103b and connection devices 150A and 150B. In one embodiment, the duration of the sterilization step is 900 s. Depending on the design of the hollow fiber membrane filter, a different duration of the sterilization step may be provided, e.g. 200 to 1400 s, or 400 to 1100 s, or 400 to 800 s.

In a further embodiment, the method according to the invention is characterized in that the introduction, in particular the passage, of sterile compressed air into/through the second compartment and the introduction, in particular the passage, of highly purified water into/through the first compartment of the hollow fiber membrane filter during the leakage test are carried out in counterflow.

In a further embodiment, the process according to the invention is characterized in that the temperature of the water passing through the first and second compartments of the hollow fiber membrane filter is 50 to 120° C., or 50 to 95° C., or 60 to 90° C., or 65 to 90° C., or 70 to 90° C.

In a further embodiment, the process according to the invention is characterized in that the sterilizing fluid is water vapor, in particular highly purified water vapor having a temperature of 105 to 150° C., which is introduced at a pressure of 1.1 to 3 bar into the first and second compartments of the hollow fiber membrane filter. In particular, pressures of 1.1 to 2.5 bar or 1.1 to 2 bar or 1.1 to 1.8 bar or 1.2 to 1.6 bar can also be used in the sterilization step. Alternative temperatures of the highly purified water vapor can range from 110 to 140° C. or from 115 to 130° C.

In a further embodiment, the method according to the invention is characterized in that between the leakage test and the sterilization step, a blow-out step is carried out with sterile compressed air, wherein the first and second compartments of the hollow fiber membrane filter are blown out with sterile compressed air.

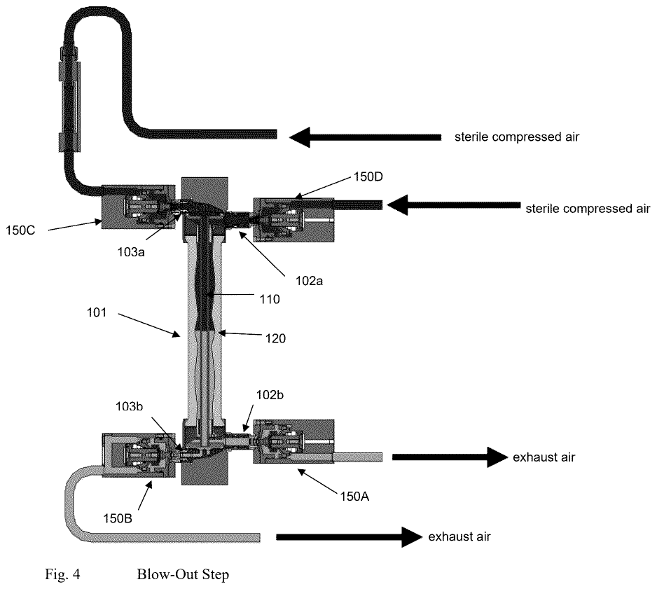

FIG. 4 shows a schematic illustration of this blow-out step. The arrangement of the hollow fiber membrane filter, the connection devices 150A, 150B, 150C, 150D in conjunction with the sterilization device (not shown in FIG. 4) is the same as in FIG. 1 to 3. FIG. 4 shows in schematic view that sterile compressed air is introduced into and passed through the first and second compartments 110, 120 of the hollow fiber membrane filter via the connection devices 150C and 150D and the fluid inlets 103a, 102a. The sterile compressed air is discharged as exhaust air from the first and second compartments 110, 120 of the hollow fiber membrane filter via ports 103b and 102b and respective connection devices 150B and 150A. The pressures of the sterile compressed air flowing into the first and second compartments 110, 120 are substantially the same, e.g. between 1 and 2 bar. The blow-out step expels residual water remaining from the leakage test from the first compartment 110 of the hollow fiber membrane filter. However, the pores of the membrane wall of the hollow fiber membranes remain preferably filled with water. The filter is thus advantageously conditioned for the subsequent sterilization step with water vapor. Depending on the design of the hollow fiber membrane filter, the blow-out step can be performed for a duration of 30 to 90 s or 40 to 80 s or 50 to 70 s.

In a further embodiment, the method according to the invention is characterized in that, prior to the leakage test, a water vapor pushing step is carried out in which water vapor is passed into, in particular is passed through, the first and the second compartment of the hollow fiber membrane filter. The water vapor pushing step is performed according to the schematic illustration shown in FIG. 3. In the water vapor pushing step, highly purified water vapor is introduced into the first compartment 110 and the second compartment 120 of the hollow fiber membrane filter, respectively, via the connection devices 150C and 150D and the fluid inlets 103a and 102a. The highly purified water vapor is provided via the sterilization device and, according to the embodiment shown, has a temperature of 123° C. and a pressure of 1.3 bar. However, depending on the design of the hollow fiber membrane filter, other pressures and temperatures may be provided. In particular, pressures of 1.1 to 3 bar or 1.1 to 2.5 bar or 1.1 to 2 bar or 1.1 to 1, 8 bar or 1.2 to 1.6 bar can also be used for the water vapor pushing step. Preferably, the pressure of the highly purified water vapor in the first and second compartments of the hollow fiber membrane filter in the water vapor pushing step is substantially the same. Alternative temperatures of the highly purified water vapor can range from 50 to 150° C., or 105 to 150° C., or from 110 to 140° C., or from 115 to 130° C. The highly purified water vapor is passed through the first compartment and the second compartment 110, 120 of the hollow fiber membrane filter, and the first compartment and the second compartment are thereby conditioned. As the highly purified water vapor passes through the first and second compartments, the highly purified water vapor cools, causing some of the highly purified water vapor to condense. In the technical process, the mixture of highly purified water vapor and condensed water is referred to as condensate and is discharged from the second compartment and the first compartment via fluid inlets 102b, 103b and connection devices 150A and 150B. Depending on the design of the hollow fiber membrane filter, a different duration of the water vapor pushing step may be provided, e.g. 40 to 140 s or 50 to 110 s or 60 to 100 s. The water vapor pushing step preconditions the hollow fiber membrane filter in terms of temperature and pressure for the subsequent sterilization step. This makes it easier to identify possible leaks in the subsequent leakage test before the sterilization step.

In the embodiment described above, the method according to the invention is further characterized in that the introduction, in particular the passing, of water vapor in the water vapor pushing step into the first and into the second compartment of the hollow fiber membrane filter is effected by the equal flow principle.

In a further embodiment, the method according to the invention is characterized in that the water vapor in the first and second compartments of the hollow fiber membrane filter has substantially the same pressure, or that the water vapor in the first compartment has a higher pressure than the water vapor in the second compartment. The pressures in the first and second compartments can be varied during the water vapor pushing step. In particular, a higher pressure in the first compartment may result in a transmembrane transfer of the highly purified water vapor from the first compartment to the second compartment. For example, blockings in the pores of the membrane can be removed and the hollow fiber membranes in the hollow fiber membrane filter are loosened, thereby increasing the separation performance of the hollow fiber membrane filter in filtration applications, such that the clearance, can be increased. It may therefore be provided that the pressure in the first compartment is 1.5 bar or 1.3 bar or 0.8 bar higher than the pressure in the second compartment, in which case the pressure in the second compartment may be 0.3 bar or 1.3 bar or 1.5 bar.

In a further embodiment of the method according to the invention, it is provided that the purging step is performed between the water vapor pushing step and the leakage test. This advantageously allows adhesions and particles loosened by the water vapor pushing step to be discharged from the hollow fiber membrane filter prior to the leakage test and sterilization step.

In a further embodiment of the method according to the invention, it is provided that a first blow-out step takes place prior to the water vapor pushing step, in which the first compartment and the second compartment of the hollow fiber membrane filter are blown out with sterile compressed air.

The first blow-out step is performed in the same way as schematically illustrated in FIG. 4. The arrangement of the hollow fiber membrane filter, the connection devices 150A, 150B, 150C, 150D in conjunction with the sterilization device (not shown in FIG. 4) is the same as in FIG. 1 to 3. FIG. 4 shows in schematic illustration that sterile compressed air is introduced into and passed through the second and first compartments 120, 110 of the hollow fiber membrane filter via the connection devices 150D and 150C and the fluid inlets 102a, 103a. The sterile compressed air is discharged as exhaust air from the first and second compartments 110, 120 of the hollow fiber membrane filter via ports 103b and 102b and respective connection devices 150B and 150A. The pressures of the sterile compressed air flowing into the first and second compartments 110, 120 are substantially equal, e.g. between 1 and 2 bar. The first blow-out step pre-dries the hollow fiber membrane filter for the subsequent water vapor pushing step. Depending on the design of the hollow fiber membrane filter, the first blow-out step can be carried out during a duration of 30 to 90 s or 40 to 80 s or 50 to 70 s.

In a further embodiment, the method according to the invention is characterized in that the sterilization step is followed by a drying step in which sterile compressed air is passed through the first compartment and the second compartment of the hollow fiber membrane filter. Technically, the drying step is carried out in the same way as one of the blow-out steps described above. However, depending on the design of the hollow fiber membrane filter, the drying step is performed for a duration of 900 to 1800 s or 1100 to 1600 s or 1200 to 1500 s or 1250 to 1400 s.

After the drying step, the hollow fiber membrane filter is in a ready-to-use condition. After the drying step, the fluid inlets 102a, 102b, 103a, 103b are closed in a germ-tight manner by appropriate caps. The fluid inlets are preferably sealed under sterile conditions in the connection devices 150A, 150B, 150C, 150D.

In a further embodiment, the process according to the invention is characterized in that the hollow fiber membranes of the hollow fiber membrane filter comprise polysulfone and polyvinylpyrrolidone or consist of polysulfone and polyvinylpyrrolidone.

In a preferred further embodiment, the hollow fiber membranes are prepared by the following method: A spinning solution consisting of 16 parts by weight of polysulfone (P3500 from Fa. Solvay), 4.4 parts by weight polyvinylpyrrolidone (K82-86 from Fa. Ashland) and 79.6 parts by weight DMAC, are processed to a homogeneous spinning mass under stirring, heating to 60° C. and degassing. The spinning mass is extruded through an annular gap nozzle to form a spinning yarn enclosing a centrally guided precipitant consisting of 35% DMAC and 65% water. The precipitant is guided inside the hollow spinning yarn. The temperature of the annular gap nozzle is 70° C. The extruded spun yarn is passed through a precipitation gap whose atmosphere has a relative humidity of 100%. The height of the precipitation gap is 200 mm and a dwell time in the precipitation gap is set to 0.4 s. The spun yarn is introduced into the precipitation bath, which consists of water at a temperature of 80° C. and is precipitated to form a hollow fiber membrane. The hollow fiber membrane is then passed through rinsing baths tempered to a temperature of 75° C. to 90° C. After that, the hollow fiber membrane is dried at a temperature between 100° C. and 150° C.

For the construction of a hollow fiber membrane filter, the obtained hollow fiber membrane is then picked up by a reel and is combined into an array of fibers. The reeled array of fibers is processed to hollow fiber membrane bundles and are used for the manufacture of the hollow fiber membrane filters.

1. A method for sterilizing a hollow fiber membrane filter having a plurality of hollow fiber membranes sealed at the ends in the housing of the hollow fiber membrane filter so that a first compartment involving the interior of the hollow fiber membranes and a second compartment involving a space between the hollow fiber membranes is formed, wherein the hollow fiber membrane filter has at least two fluid inlets connected to the first compartment and has at least two fluid inlets connected to the second compartment, and wherein the fluid inlets are configured to be connected to a sterilization device, said method comprising at least the steps of:
a) rinsing the hollow fiber membrane filter with a fluid wherein the rinsing fluid is passed through the first and second compartments of the hollow fiber membrane filter via a selection of the fluid inlets,
b) carrying out a leakage test,
c) sterilizing the hollow fiber membrane filter with a sterilizing fluid, wherein the sterilizing fluid is passed through the first and second compartments of the hollow fiber membrane filter via a selection of the fluid inlets,
and wherein the leakage test is carried out prior to the sterilizing step.

2. The method according to claim 1, wherein in the leakage test, sterile compressed air is introduced into the second compartment of the hollow fiber membrane filter, and highly purified water is introduced into the first compartment of the hollow fiber membrane filter, and the pressure in the second compartment is higher than in the first compartment.

3. The method according to claim 2, wherein the introduction of sterile compressed air into the second compartment and the introduction of highly purified water into the first compartment of the hollow fiber membrane filter are carried out in counterflow principle.

4. The method according to claim 1, wherein the temperature of the water passed through the first and second compartments of the hollow fiber membrane filter is 50 to 120° C.

5. The method according to claim 1, wherein the sterilizing fluid is water vapor having a temperature of 105 to 150° C., which is introduced into the first and second compartments of the hollow fiber membrane filter at a pressure of 1.1 to 3 bar.

6. The method according to claim 1, wherein between the leakage test and the sterilizing step, a blow-out step with sterile compressed air is caried out, wherein the first and second compartments of the hollow fiber membrane filter are blown out with sterile compressed air.

7. The method according to claim 1, wherein prior to the leakage test, a water vapor pushing step is carried out in which water vapor is passed into the first and into the second compartments of the hollow fiber membrane filter.

8. The method according to claim 7, wherein the introduction, of water vapor into the first and into the second compartment of the hollow fiber membrane filter is effected by the equal flow principle.

9. The method according to claim 7, wherein the water vapor has a temperature of 100 to 150° C. and a pressure of 1.1 to 3 bar.

10. The method of claim 7, wherein the water vapor in the first and second compartments of the hollow fiber membrane filter has substantially the same pressure, or that the water vapor in the first compartment has a higher pressure than the water vapor in the second compartment.

11. The method according to claim 7, wherein the rinsing step takes place between the water vapor pushing step and the leakage test before sterilization.

12. The method of claim 7, wherein a first blow-out step takes place prior to the water vapor pushing step, in which the first compartment and the second compartment of the hollow fiber membrane filter are blown out with sterile compressed air.

13. The method according to claim 1, wherein the sterilization step is followed by a drying step in which sterile compressed air is passed through the first compartment and the second compartment of the hollow fiber membrane filter.

14. The process according to claim 1, wherein the hollow fiber membranes comprise polysulfone and polyvinylpyrrolidone or consist of polysulfone and polyvinylpyrrolidone.

15. The method of claim 1, wherein said fluid is water.

16. The method of claim 1, wherein said sterilizing fluid is heated water or water vapor.

17. The method according to claim 1, wherein in the leakage test, sterile compressed air is passed through the second compartment of the hollow fiber membrane filter, and highly purified water is passed through the first compartment of the hollow fiber membrane filter, and the pressure in the second compartment is higher than in the first compartment.

18. The method according to claim 2, wherein the introduction is a passage of sterile compressed air into the second compartment and the introduction is a passage of highly purified water into the first compartment of the hollow fiber membrane filter are carried out in counterflow principle.

19. The method according to claim 1, wherein prior to the leakage test, a water vapor pushing step is carried out in which water vapor is passed through into the first and into the second compartments of the hollow fiber membrane filter.

20. The method according to claim 7, wherein the introduction is a passing of water vapor into the first and into the second compartment of the hollow fiber membrane filter is effected by the equal flow principle.
